SEQUENCE LISTING (15 sequences)

SEQ ID NO: 1 - protein, 210 aa, Homo sapiens
MWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 2 - protein, 130 aa, Homo sapiens
SGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 3 - protein, 80 aa, Homo sapiens
MWTLGRRAVAGLLASPSPAQAQTLTRVPRPAELAPLCGRRGLRTDIDATCTPRRASSNQRGLNQIWNVKKQSVYLMNLRK

SEQ ID NO: 4 - protein, 11 aa, synthetic construct
  REGION 1..11: note = source = /note="Description of Artificial Sequence: Synthetic peptide"
YGRKKRRQRRR

SEQ ID NO: 5 - protein, 30 aa, synthetic construct
  REGION 1..30: note = source = /note="Description of Artificial Sequence: Synthetic polypeptide"
GWTLNSAGYLLGPHAVGNHRSFSDKNGLTS

SEQ ID NO: 6 - protein, 14 aa, synthetic construct
  REGION 1..14: note = source = /note="Description of Artificial Sequence: Synthetic peptide"
INLKALAALAKKIL

SEQ ID NO: 7 - protein, 27 aa, synthetic construct
  REGION 1..27: note = source = /note="Description of Artificial Sequence: Synthetic peptide"
GWTLNSAGYLLGKINLKALAALAKKIL

SEQ ID NO: 8 - protein, 16 aa, synthetic construct
  REGION 1..16: note = source = /note="Description of Artificial Sequence: Synthetic peptide"
RQIKIWFQNRRMKWKK

SEQ ID NO: 9 - protein, 9 aa, synthetic construct
  REGION 1..9: note = source = /note="Description of Artificial Sequence: Synthetic peptide"
RRRRRRRRR

SEQ ID NO: 10 - protein, 34 aa, synthetic construct
  REGION 1..34: note = source = /note="Description of Artificial Sequence: Synthetic polypeptide"
DAATATRGRSAASRPTERPRAPARSASRPRRPVE

SEQ ID NO: 11 - protein, 224 aa, synthetic construct
  REGION 1..224: note = source = /note="Description of Artificial Sequence: Synthetic polypeptide"
MYGRKKRRQRRRGGMWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 12 - protein, 8 aa, synthetic construct
  REGION 1..8: note = source = /note="Description of Artificial Sequence: Synthetic peptide"
GGMWTLGR

SEQ ID NO: 13 - protein, 17 aa, synthetic construct
  REGION 1..17: note = source = /note="Description of Artificial Sequence: Synthetic peptide"
SGTLGHPGSLDETTYER

SEQ ID NO: 14 - protein, 12 aa, synthetic construct
  REGION 1..12: note = source = /note="Description of Artificial Sequence: Synthetic peptide"
LGGDLGTYVINK

SEQ ID NO: 15 - protein, 12 aa, synthetic construct
  REGION 1..12: note = source = /note="Description of Artificial Sequence: Synthetic peptide"
MYGRKKRRQRRR

(19) United States
(12) Patent Application Publication
(10) Pub. No.: US 2026/0140121 A1
(43) Pub. Date: May 21, 2026

(54) METHODS OF QUANTIFYING FRATAXIN AND FRATAXIN FUSION PROTEINS

(71) Applicants: Larimar Therapeutics, Inc., Bala Cynwyd, PA (US); The United States of America, as represented by the Secretary, Department of Health and Human servic, Bethesda, MD (US)

(72) Inventors: Joan David Bettoun, Houston, TX (US); Erik Johan Wagner, Sterling, VA (US); Xin Xu, Potomac, MD (US); Jean-Nicholas Mess, Laval (CA); Amy Qiu Wang, Poolesville, MD (US)

(21) Appl. No.: 19/456,394

(22) Filed: Jan. 22, 2026

Related U.S. Application Data
(63) Continuation of application No. 17/631,414, now Pat. No. 12,566,178.
(60) Provisional application No. 62/880,068, filed on Jul. 29, 2019.
(60) Provisional application No. 62/936,293, filed on Nov. 15, 2019.

(51) Int. Cl. G01N 33/68

(52) U.S. Cl. CPC G01N 33/6848

(57) ABSTRACT

The present disclosure provides methods for determining the amount of FXN or FXN fusion protein in a sample, e.g., a tissue sample, by using mass spectrometry.

### RELATED APPLICATIONS

This application is a continuation of U.S. patent application Ser. No. 17/631,414, filed on Jan. 28, 2022; which is a 35 U.S.C. § 371 national stage filing of International Application No. PCT/US2020/044118, filed on Jul. 29, 2020; which claims priority to U.S. Provisional Patent Application No. 62/936,293, filed on Nov. 15, 2019 and U.S. Provisional Patent Application No. 62/880,068, filed on Jul. 29, 2019. The entire contents of each of the foregoing applications are hereby incorporated herein by reference.

### REFERENCE TO ELECTRONIC SEQUENCE

The application contains a Sequence Listing which has been submitted electronically in .XML format and is hereby incorporated by reference in its entirety. Said. XML copy, created on Jan. 22, 2026, is named “130197-00903.xml” and is 19,954 bytes in size. The sequence listing contained in this .XML file is part of the specification and is hereby incorporated by reference herein in its entirety.

### INTRODUCTION

Friedreich's Ataxia (FRDA) is a rare genetic, progressive neurodegenerative disorder caused by a mutation in a gene encoding frataxin (FXN). FXN is an essential and phylogenetically conserved protein that is found in cells throughout the body, with the highest levels in the heart, spinal cord, liver, pancreas, and skeletal muscle. FXN is encoded in the nucleus, expressed in the cytoplasm and imported into the mitochondria where it is processed to the mature form. In humans, the 210-amino acid full-length hFXN (hFXN₁₋₂₁₀, 23.1 kDa) contains a typical mitochondrial targeting sequence (MTS) at the amino terminus that is processed in a 2-step cleavage by the mitochondrial matrix processing peptidase (MPP) as it is imported into the mitochondrial matrix. The resulting protein is a 130-amino acid, 14.2 kDa mature hFXN protein (hFXN₈₁₋₂₁₀).

TAT-GG-FXN is a 24.9 kDa fusion protein currently under investigation as an FXN replacement therapy to restore functional levels of FXN in the mitochondria of FRDA patients. TAT-GG-FXN includes the HIV-TAT peptide linked to the N-terminus of the full-length hFXN protein. The mechanism of action of TAT-GG-FXN relies on the cell-penetrating ability of the HIV-TAT peptide to deliver the TAT-GG-FXN into cells and the subsequent processing into mature hFXN after translocation into the mitochondria. TAT-GG-FXN (SEQ ID NO: 11) is described in U.S. Provisional Patent Application No. 62/891,029, the entire contents of each of which are hereby incorporated herein by reference.

To facilitate development of FXN replacement therapies, new reliable methods are needed to accurately and precisely measure level of FXN and FXN fusion proteins in various biological fluids and tissues of a subject.

### SUMMARY OF THE INVENTION

In one aspect, the present disclosure provides a method for determining the amount of unprocessed frataxin (FXN) fusion protein in a sample, said method comprising determining the amount of a peptide in said sample by using mass spectrometry (MS),
wherein said peptide comprises, or consists of, the amino acid sequence GGMWTLGR (SEQ ID NO: 12); and
wherein said unprocessed FXN fusion protein comprises, or consists of, from N-terminus to C-terminus, a cell penetrating peptide (CPP); a mitochondrial targeting sequence (MTS) comprising, or consisting of, SEQ ID NO: 3; and FXN comprising, or consisting of, SEQ ID NO: 2.

In one embodiment, said sample is obtained from a subject being administered the unprocessed FXN fusion protein.

In one embodiment, said CPP comprises, or consists of, HIV-TAT (SEQ ID NO: 4) or HIV-TAT+M (SEQ ID NO: 15).

In one embodiment, said unprocessed FXN fusion protein is TAT-GG-FXN (SEQ ID NO: 11).

In one embodiment, said method comprises purifying by immunocapture the unprocessed FXN fusion protein from said sample prior to MS.

In one embodiment, said method comprises the following steps:
a) purifying by immunocapture the unprocessed FXN fusion protein from said sample, thereby obtaining immunocaptured complexes comprising the unprocessed FXN fusion protein;
b) subjecting said immunocaptured complexes comprising the unprocessed FXN fusion protein to digestion by trypsin, thereby producing the peptide comprising, or consisting of, SEQ ID NO: 12; and
c) determining the amount of the peptide comprising, or consisting of, SEQ ID NO: 12 by liquid chromatography and tandem mass spectrometry (LC/MS-MS).

In one embodiment, in part c) the liquid chromatography (LC) comprises reversed-phase chromatography.

In one embodiment, tandem mass spectrometry (MS-MS) comprises monitoring the transition 439.2→632.3.

In one embodiment, the tandem mass spectrometry (MS-MS) comprises monitoring the transition 439.2→446.3.

In one embodiment, the immunocapture is carried out using a binding protein comprising an antigen binding domain that specifically binds to the FXN fusion protein.

In one embodiment, said binding protein specifically binds to the FXN present in the FXN fusion protein.

In one embodiment, said binding protein specifically binds to the CPP present in the FXN fusion protein.

In one embodiment, the FXN fusion protein comprises HIV-TAT (SEQ ID NO: 4) or HIV-TAT+M (SEQ ID NO: 15) and wherein said binding protein specifically binds to the HIV-TAT.

In one embodiment, the sample is a tissue sample.

In one embodiment, the tissue sample comprises a buccal swab.

In one embodiment, the tissue sample comprises a skin biopsy.

In one embodiment, the sample is derived from the blood of the subject.

In one embodiment, the sample comprises platelets.

In one embodiment, the sample is a plasma sample.

In one embodiment, the method has a precision as measured by % coefficient of variation (% CV) of about 20% or less.

In one embodiment, the method has a precision as measured by % CV of about 15% or less, about 14% or less, about 13% or less, about 12% or less, about 11% or less, about 10% or less, about 9% or less, about 8% or less, about 7% or less, about 6% or less, about 5% or less, about 4% or less, about 3% or less, about 2% or less or about 1% or less.

In one embodiment, the method has an accuracy of about 80% to about 120%.

In one embodiment, the method is linear for concentration of unprocessed FXN fusion protein ranging from about 0.250 ng/mL to about 25.000 ng/mL.

In another aspect, the present disclosure provides a method of monitoring processing of unprocessed FXN fusion protein in a sample, said method comprising:
obtaining at least two temporally separated samples from said subject; and
determining the amount of unprocessed FXN fusion protein in said temporally separated samples using the methods of the present disclosure.

In one embodiment, the sample is derived from a subject being administered the unprocessed FXN fusion protein.

In yet another aspect, the present disclosure provides a method of adjusting a dosing schedule for administering unprocessed FXN fusion protein, said method comprising:
obtaining at least two temporally separated samples from the subject being administered unprocessed FXN fusion protein;
determining the amount of unprocessed FXN fusion protein in said temporally separated samples using the methods of the present disclosure; and
adjusting the dose of unprocessed FXN fusion protein being administered to the subject and/or dosing frequency of the unprocessed FXN fusion protein being administered to the subject based on the temporal changes in the amount of unprocessed FXN fusion protein observed in the subject.

In still another aspect, the present disclosure provides a method for determining the amount of unprocessed FXN fusion protein, e.g., TAT-GG-FXN (SEQ ID NO: 11), in a sample, said method comprising determining the amount of at least one peptide in said sample by using MS,
wherein said at least one peptide is selected from the group consisting of:
   a) a peptide comprising, or consisting of, the amino acid sequence SGTLGHPGSLDETTYER (SEQ ID NO: 13); and
   b) a peptide comprising, or consisting of, the amino acid sequence LGGDLGTYVINK (SEQ ID NO: 14);
wherein said method comprises purifying by immunocapture the unprocessed TAT-GG-FXN from said sample prior to MS;
wherein said immunocapture is carried out using a binding protein comprising an antigen binding domain, wherein said binding protein is capable of binding a TAT protein transduction domain.

In one embodiment, said sample is obtained from a subject being administered the unprocessed TAT-GG-FXN (SEQ NO: 11).

In one embodiment, said method comprises the following steps:
a) purifying by immunocapture the unprocessed FXN fusion protein, e.g., TAT-GG-FXN from said sample, thereby obtaining immunocaptured complexes comprising the unprocessed FXN fusion protein, e.g., TAT-GG-FXN;
b) subjecting said immunocaptured complexes comprising the unprocessed FXN fusion protein, e.g., TAT-GG-FXN to digestion by a protease, e.g., trypsin, thereby producing the peptide comprising, or consisting of, SEQ ID NO: 13 and/or the peptide comprising, or consisting of, SEQ ID NO: 14; and
c) determining the amount of the peptide comprising, or consisting of, SEQ ID NO: 13 and/or the peptide comprising, or consisting of, SEQ ID NO: 14 by LC/MS-MS.

In one embodiment, in part c) the LC comprises reversed-phase chromatography.

In one embodiment, the MS-MS comprises monitoring the transition 607.3→669.3 for the peptide comprising, or consisting of, SEQ ID NO: 13 and the transition 625.3→794.4 for the peptide comprising, or consisting of, SEQ ID NO: 14.

In one embodiment, the sample is a tissue sample.

In one embodiment, the tissue sample comprises a buccal swab.

In one embodiment, the tissue sample comprises a skin biopsy.

In one embodiment, the sample is derived from the blood of the subject.

In one embodiment, the sample comprises platelets.

In one embodiment, the sample is a plasma sample.

In one embodiment, the method has a precision as measured by % coefficient of variation (% CV) of 20% or less;

In one embodiment, the method has an accuracy of about 80% to about 120%.

In one embodiment, the method is linear for concentrations of unprocessed TAT-GG-FXN ranging from 0.250 ng/mL to 25.000 ng/mL.

In another aspect, the present disclosure provides a method for determining the amount of FXN in a tissue sample, said method comprising determining the amount of at least one peptide derived from said FXN in said sample by using MS,
wherein said at least one peptide is selected from the group consisting of:
   a) a peptide comprising, or consisting of, the amino acid sequence SGTLGHPGSLDETTYER (SEQ ID NO: 13); and
   b) a peptide comprising, or consisting of, the amino acid sequence LGGDLGTYVINK (SEQ ID NO: 14);
wherein the method is characterized by at least one of the following:
   the method has a precision as measured by % coefficient of variation (% CV) of 20% or less; and
   the method has an accuracy of about 80% to about 120%.

In one embodiment, said sample is obtained from a subject.

In one embodiment, said subject is being administered unprocessed FXN fusion protein.

In one embodiment, said unprocessed FXN fusion protein comprises, or consists of, from N-terminus to C-terminus, a cell penetrating peptide (CPP), a mitochondrial targeting sequence (MTS) of SEQ ID NO: 3 and FXN.

In one embodiment, said CPP comprises, or consists of, HIV-TAT (SEQ ID NO: 4) or HIV-TAT+M (SEQ ID NO: 15).

In one embodiment, said unprocessed FXN fusion protein is TAT-GG-FXN (SEQ ID NO: 11).

In one embodiment, said method comprises purifying by immunocapture the FXN from said sample prior to MS.

In one embodiment, said method comprises the following steps:
a) purifying by immunocapture the FXN from said sample, thereby obtaining immunocaptured complexes comprising FXN;
b) subjecting said immunocaptured complexes comprising FXN to digestion by a protease, e.g., trypsin, thereby producing the peptide comprising, or consisting of, SEQ ID NO: 13 and/or the peptide comprising, or consisting of, SEQ ID NO: 14; and
c) determining the amount of at least one peptide selected from the group consisting of the peptide comprising, or consisting of, SEQ ID NO: 13 and/or the peptide comprising, or consisting of, SEQ ID NO: 14 by LC/MS-MS.

In one embodiment, in part c) the LC comprises reversed-phase chromatography.

In one embodiment, the tandem mass spectrometry (MS-MS) comprises monitoring the transition 607.3→669.3 for the peptide comprising, or consisting of, SEQ ID NO: 13 and the transition 625.3→794.4 for the peptide comprising, or consisting of, SEQ ID NO: 14.

In one embodiment, the immunocapture is carried out using a binding protein comprising an antigen binding domain that specifically binds FXN.

In one embodiment, said FXN is a part of an FXN fusion protein also comprising HIV-TAT (SEQ ID NO: 4) or HIV-TAT+M (SEQ ID NO: 15) and wherein said binding protein specifically binds the HIV-TAT.

In one embodiment, the tissue sample comprises a buccal swab.

In one embodiment, the tissue sample comprises a skin biopsy.

In another aspect, the present disclosure provides a method for determining the amount of FXN in a skin sample, said method comprising determining the amount of at least one peptide derived from said FXN in said skin sample by using MS,
wherein said at least one peptide is selected from the group consisting of:
   a) a peptide comprising, or consisting of, the amino acid sequence GGMWTLGR (SEQ ID NO: 12);
   b) a peptide comprising, or consisting of, the amino acid sequence SGTLGHPGSLDETTYER (SEQ ID NO: 13); and
   c) a peptide comprising, or consisting of, the amino acid sequence LGGDLGTYVINK (SEQ ID NO: 14).

In one embodiment, said skin sample is obtained from a subject.

In one embodiment, said subject is being administered unprocessed FXN fusion protein.

In one embodiment, said unprocessed FXN fusion protein comprises, or consists of, from N-terminus to C-terminus, a cell penetrating peptide (CPP), a mitochondrial targeting sequence (MTS) of SEQ ID NO: 3 and FXN, e.g., FXN of SEQ ID NO: 2.

In one embodiment, said CPP comprises, or consists of, HIV-TAT (SEQ ID NO: 4) or HIV-TAT+M (SEQ ID NO: 15).

In one embodiment, said unprocessed FXN fusion protein is TAT-GG-FXN (SEQ ID NO: 11).

In one embodiment, said method comprises purifying by immunocapture the FXN from said skin sample prior to MS.

In one embodiment, said method comprises the following steps:
a) purifying by immunocapture FXN from said skin sample, thereby obtaining immunocaptured complexes comprising FXN;
b) subjecting said immunocaptured complexes comprising FXN to digestion by a protease, e.g., trypsin, thereby producing the peptide comprising, or consisting of, SEQ ID NO: 12 and/or the peptide comprising, or consisting of, SEQ ID NO: 13 and/or the peptide comprising, or consisting of, SEQ ID NO: 14; and
c) determining the amount of at least one peptide selected from the group consisting of the peptide comprising, or consisting of, SEQ ID NO: 12 and/or the peptide comprising, or consisting of, SEQ ID NO: 13 and/or the peptide comprising, or consisting of, SEQ ID NO: 14 by LC/MS-MS.

In one embodiment, said immunocapture is carried out using a binding protein comprising an antigen binding domain that specifically binds FXN.

In one embodiment, said FXN is a part of an FXN fusion protein also comprising HIV-TAT (SEQ ID NO: 4) or HIV-TAT+M (SEQ ID NO: 15) and wherein said binding protein specifically binds the HIV-TAT.

### BRIEF DESCRIPTION OF THE DRAWINGS

FIG. 1 is an illustration of the procedure used for the preparation of biological samples for quantifying TAT-GG-FXN (SEQ ID NO: 11).

FIG. 2 is an illustration of sample processing for hybrid LC-MS/MS analysis. FIG. 2 discloses SEQ ID NOS 12-14, respectively, in order of appearance.

FIG. 3 is a series of graphs showing the concentration of TAT-GG-FXN (SEQ ID NO: 11) levels in monkey tissues (N=6) following repeated subcutaneous (SC) administration at 15 mg/kg BID for 14 days. Data is expressed at pg of TAT-GG-FXN per μg of total protein.

FIG. 4 is a series of representative chromatograms of TAT-GG-FXN tryptic peptides in monkey tissues prior to dosing (Day 1) and following repeated SC administration at 15 mg/kg BID for 14 days.

### DETAILED DESCRIPTION OF THE INVENTION

The present disclosure provides methods for determining the amount of frataxin (FXN) or an FXN fusion protein in a sample using mass spectrometry (MS). FXN is a protein which is associated with the disorder Friedreich's Ataxia (FRDA). FRDA is a genetic, progressive neurodegenerative disorder caused by a mutation in a gene encoding FXN. Methods of the present disclosure may be used, e.g., to monitor the levels of FXN in a subject, e.g., a subject who is being administered FXN as a protein replacement therapy for treating FRDA. Methods of the present invention may also be used to monitor processing of an FXN fusion protein, e.g., TAT-GG-FXN (SEQ ID NO: 11), after the FXN fusion protein is added to an in vitro sample or is administered to a subject. Methods of the present disclosure may also be used to monitor levels of FXN or an FXN fusion protein in a subject, e.g., subject who is being administered an FXN fusion protein, and to adjust the dose or the dosing frequency of the FXN fusion protein.

### FXN and FXN Fusion Protein

FXN is an essential and phylogenetically conserved protein that is found in cells throughout the body, with the highest levels in the heart, spinal cord, liver, pancreas, and skeletal muscle. FXN is encoded in the nucleus, expressed in the cytoplasm and imported into the mitochondria where it is processed to the mature form. In humans, the 210-amino acid full-length hFXN (hFXN₁₋₂₁₀, 23.1 kDa) contains a typical mitochondrial targeting sequence (MTS) at the amino terminus that is processed in a 2-step cleavage by the mitochondrial matrix processing peptidase (MPP) as it is imported into the mitochondrial matrix. The resulting protein is a 130-amino acid, 14.2 kDa mature hFXN protein (hFXN₈₁₋₂₁₀). Sequences of the full-length hFXN and mature hFXN are shown in Table 1 below.

TABLE 1
Sequences of the full-length hFXN and mature hFXN.

| SEQ ID NO. | Protein | Amino Acid Sequence |
|---|---|---|
| 1 | Full-length | MWTLGRRAVAGLLASPSPAQAQTLTRVPRPAELAPLCGR |
| | hFXN | RGLRTDIDATCTPRRASSNQRGLNQIWNVKKQSVYLMNL |
| | hFXN₁₋₂₁₀ | RKSGTLGHPGSLDETTYERLAEETLDSLAEFFEDLADKPY |
| | | TFEDYDVSFGSGVLTVKLGGDLGTYVINKQTPNKQIWLS |
| | | SPSSGPKRYDWTGKNWVYSHDGVSLHELLAAELTKALK |
| | | TKLDLSSLAYSGKDA |
| 2 | Mature | SGTLGHPGSLDETTYERLAEETLDSLAEFFEDLADKPYTF |
| | hFXN | EDYDVSFGSGVLTVKLGGDLGTYVINKQTPNKQIWLSSP |
| | hFXN₈₁₋₂₁₀ | SSGPKRYDWTGKNWVYSHDGVSLHELLAAELTKALKTK |
| | | LDLSSLAYSGKDA |

The full-length hFXN (SEQ ID NO: 1) comprises mature hFXN (SEQ ID NO: 2) and a mitochondrial targeting sequence (MTS) having the amino acid sequence

(SEQ ID NO: 3)
MWTLGRRAVAGLLASPSPAQAQTLTRVPRPAELAPLCGRRGLRTDIDATCT
PRRASSNQRGLNQIWNVKKQSVYLMNLRK.

In some embodiments, the FXN that is measured by the methods of the present disclosure may be a mammalian FXN. In some embodiments, the FXN that is measured by the methods of the present disclosure may be a human FXN (hFXN). In some embodiments, the FXN that is measured by the methods of the present invention may be a monkey FXN, e.g., FXN from a cynomolgus macaque.

In some aspects, the FXN that is quantified by the methods provided by the present disclosure may be an endogenous FXN, i.e., FXN that is naturally present in an in vitro sample, e.g., a biological sample from a subject, or in a subject. In other aspects, the FXN that is quantified by the methods provided by the present disclosure may be an exogenous FXN, i.e., FXN that has been added to an in vitro sample, or that has been administered to a subject, e.g., and is thereby present in a biological sample from the subject. An exogenous FXN may be comprised in an FXN fusion protein, e.g., TAT-GG-FXN (SEQ ID NO: 11).

As used herein, the term “FXN fusion protein” refers to an artificial polypeptide that comprises FXN, e.g., full-length hFXN (SEQ ID NO: 1) or mature hFXN (SEQ ID NO: 2). In some embodiments, the FXN fusion protein also comprises a cell penetrating peptide (CPP).

The term “cell penetrating peptide” or “CPP”, as used herein, refers to a short peptide sequence, typically between 5 and 30 amino acids long, that can facilitate cellular intake of various molecular cargo, such as proteins. Within the context of the present invention, a CPP present in an FXN fusion protein facilitates the delivery of the FXN fusion protein into a cell, e.g., a recipient cell.

CPPs may be polycationic, i.e., have an amino acid composition that either contains a high relative abundance of positively charged amino acids, such as lysine or arginine. CCPs may also be amphipathic, i.e., have sequences that contain an alternating pattern of polar/charged amino acids and non-polar, hydrophobic amino acids. CPPs may also be hydrophobic, i.e., contain only apolar residues with low net charge, or have hydrophobic amino acid groups that are crucial for cellular uptake.

A CPP that may be comprised in the FXN fusion protein useful in the context of the present invention may be any CPP known to a person skilled in the art. For example, the CPP may be any CPP listed in the Database of Cell-Penetrating Peptides CPPsite 2.0, the entire contents of which are hereby incorporated herein by reference. For examples, CPPs useful in the context of the present invention may a cell penetrating peptide derived from a protein selected from the group consisting of HIV Trans-Activator of Transcription peptide (HIV-TAT), galanin, mastoparan, transportan, penetratin, polyarginine, or VP22. In some embodiments, the CPP comprises a transduction domain of TAT protein comprising amino acids 47-57 of the 86 amino acid full length HIV-TAT protein (which 11 amino acid peptide may also be referred to herein as “HIV-TAT”; SEQ ID NO:4). In one embodiment, the CPP consists of HIV-TAT (SEQ ID NO:4). In some embodiments, the CPP comprises amino acids 47-57 of the 86 amino acid full length HIV-TAT protein with a methionine added at the amino terminus for initiation (12 AA; “HIV-TAT+M”): MYGRKKRRQRRR (SEQ ID NO: 15). Table 1 below lists amino acid sequences of exemplary CPPs.

TABLE 1
Exemplary CPPs and corresponding sequences

| SEQ | | |
|---|---|---|
| ID | | |
| NO. | CPP | Amino Acid Sequence |
| 4 | HIV-TAT | YGRKKRRQRRR |
| 15 | HIV-TAT + M | MYGRKKRRQRRR |
| 5 | Galanin | GWTLNSAGYLLGPHAVGNHRSFSDKNGLTS |
| 6 | Mastoparan | INLKALAALAKKIL-NH₂ |
| 7 | Transportan | GWTLNSAGYLLGKINLKALAALAKKIL |
| 8 | Penetratin | RQIKIWFQNRRMKWKK |
| 9 | Polyarginine | RRRRRRRRR |
| 10 | VP22 | DAATATRGRSAASRPTERPRAPARSASRPR |
| | | RPVE |

In some embodiments, the CPP comprised in the FXN fusion protein is HIV-TAT (SEQ ID NO: 4) or HIV-TAT+M (SEQ ID NO: 15). In some embodiments, the FXN fusion protein comprises full-length FXN, e.g., SEQ ID NO: 1, and HIV-TAT, e.g., SEQ ID NO: 4 or HIV-TAT+M (SEQ ID NO: 15), as CPP.

In some embodiments, in FXN fusion proteins of the present disclosure, the CPP may be fused together with the FXN, e.g., full-length FXN, via a linker to form a single polypeptide chain. In one embodiment, the linker may comprise the amino acid sequence GG. In one embodiment, the linker consists of the amino acid sequence GG. In some embodiments, the CPP is located at the N-terminus and the FXN is located at the C-terminus. In some embodiments, the CPP is located at the C-terminus and the FXN is located at the N-terminus. It will be appreciated that, in some embodiments, the methods provided herein may be used to detect an FXN fusion protein comprising any CPP and FXN, e.g., full-length FXN, wherein the CPP and FXN are fused via a GG linker.

In some embodiments, the FXN fusion protein comprises the following amino acid sequence (224 amino acids): MYGRKKRRQRRRGGMWTLGRRAVAGLLASPSPAQAQTLTR VPRPAELAPLCGRRG LRTDIDATCTPRRASSNQRGLNQIWNVKKQSVYLMNLRKSGTLGHPGSLDETTYERL AEETLDSLAEFFEDLADKPYTFEDYDVSFGSGVLTVKLGGDLGTYVINKQTPNKQIW LSSPSSGPKRYDWTGKNWVYSHDGVSLHELLAAELTKALKTKLDLSSLAYSGKDA (SEQ ID NO: 11). In some embodiments, the FXN fusion protein consists of the amino acid sequence of SEQ ID NO: 11, i.e., TAT-GG-FXN. TAT-GG-FXN is further described in U.S. Provisional Patent Application Nos. 62/891,029, the entire contents of which are hereby incorporated herein by reference.

In some aspects, the CPP, e.g., HIV-TAT, that is present in an FXN fusion protein of the present disclosure facilitates delivery of the FXN fusion protein into a cell, e.g., a cell that may be present in vitro, ex vivo, or in a subject. Once inside the cell, the FXN fusion protein may be processed by cellular machinery to remove the CPP, e.g., HIV-TAT, from the FXN. Accordingly, the term “unprocessed FXN fusion protein”, as used herein, refers to an intact FXN fusion protein, i.e., the full length FXN fusion protein, or the FXN fusion protein from which CPP, e.g., HIV-TAT, has not been removed. Such an FXN fusion protein may be an FXN fusion protein that has not been delivered into a cell. For example, an unprocessed FXN fusion protein may be present in the circulation, e.g., in the plasma, of a subject following administration to the subject of the FXN fusion protein. In some embodiments, an FXN fusion protein may be an FXN fusion protein that has been delivered to a cell but has not been processed by cellular machinery to remove the CPP, e.g., HIV-TAT. In some embodiments, the unprocessed FXN fusion protein comprises the amino acid sequence of SEQ ID NO: 11. In some embodiments, the unprocessed FXN fusion protein consists of the amino acid sequence of TAT-GG-FXN (SEQ ID NO: 11).

### Mass Spectrometry-Based Methods for Quantifying FXN and FXN Fusion Proteins

The present disclosure provides mass spectrometry-based methods for determining the amount of FXN or FXN fusion protein, e.g., unprocessed frataxin (FXN) fusion protein, such as TAT-GG-FXN (SEQ ID NO: 11), in a sample. In some embodiments, the sample may be an in vitro sample. In some embodiments, the sample may be a biological sample, e.g., comprising biological fluid or tissue. In some embodiments, the biological sample (e.g., comprising biological fluid or tissue) is derived from a subject, e.g., a subject with FRDA and/or a subject being administered the unprocessed FXN fusion protein.

In some aspects, the amount of FXN or FXN fusion protein in a sample determined using the methods of the present disclosure is an amount that is not normalized. In other aspects, the amount of FXN or FXN fusion protein determined using the methods of the present disclosure are normalized, e.g., normalized to the total amount of protein present in the sample. The amount of total protein present in a sample may be determined by any method known in the art for determining the amount of total protein, e.g., the BCA assay.

In some aspects, methods of the present disclosure are characterized by a high precision and accuracy of measurement of the amount of FXN or FXN fusion protein. For example, methods provided by the present invention may be characterized by a precision as measured by % coefficient of variation (% CV) of about 20% or less, e.g., about 15% or less, about 14% or less, about 13% or less, about 12% or less, about 11% or less, about 10% or less, about 9% or less, about 8% or less, about 7% or less, about 6% or less, about 5% or less, about 4% or less, about 3% or less, about 2% or less or about 1% or less. In some aspects, methods provided by the present invention may be characterized by % CV of 10% of less.

In some aspects, methods of the present disclosure may be characterized by an accuracy of about 80% to about 120%, e.g., about 80% to about 110%, or about 80% to about 100%, or about 85% to about 120%, or about 85% to about 110%, or about 85% to about 100%, or about 90% to about 120%, or about 90% to about 110%, or about 90% to about 100%, or about 95% to about 120%, or about 95% to about 110%, or about 95% to about 100%

In some aspects, methods of the present disclosure, when used to measure the amount of an FXN fusion protein, may be linear for concentrations of unprocessed FXN fusion protein ranging from about 0.250 ng/ml to about 25.000 ng/mL.

In some aspects, methods of the present disclosure, when used to measure the amount of TAT-GG-FXN (SEQ ID NO: 11), may be linear for concentrations of unprocessed TAT-GG-FXN ranging from about 0.250 ng/mL to about 25.000 ng/mL.

In some embodiments, methods of the present disclosure may comprise purifying by immunocapture FXN or FXN fusion protein present in a sample, thereby obtaining immunocaptured complexes comprising the FXN or FXN fusion protein; subjecting the immunocaptured complexes to digestion by a protease to obtain peptides derived from FXN or FXN fusion protein; and analyzing the peptides derived from FXN or FXN fusion protein by liquid chromatography and tandem mass spectrometry (LC-MS/MS) to determine the amount of FXN or FXN fusion protein present in a sample. In some embodiments, the protease may be selected from the group consisting of trypsin, chymotrypsin, LysC, LysN, AspN, GluC and ArgC. In one embodiment, the protease is trypsin.

In some aspects, methods of the present disclosure comprise detecting peptides derived from FXN or FXN fusion protein after digestion of the immunocaptured complexes with a protease, e.g., trypsin, i.e., tryptic peptides derived from FXN and/or FXN fusion protein. The tryptic peptides that may be monitored and/or quantified in the methods of the present disclosure are summarized in Table 2 below.

TABLE 2
Peptides quantified and/or monitored in the methods of the invention

| Peptide | | SEQ | |
|---|---|---|---|
| Name | Amino Acid Sequence | ID NO. | Characteristics |
| Linker | GGMWTLGR | 12 | Unique to TAT-GG-FXN; |
| Peptide | | | Linker between TAT and MTS |
| (Peptide | | | sequences; |
| GGM) | | | Not present in TAT-GG-FXN mature |
| | | | form |
| Peptide | SGTLGHPGSLDETTYER | 13 | N-terminal peptide of the mature TAT- |
| SGT | | | GG-FXN (i.e., of the mature hFXN); |
| | | | Not found in the monkey proteome; |
| | | | Surrogate peptide for TAT-GG-FXN |
| | | | quantitation |
| Peptide | LGGDLGTYVINK | 14 | Peptide from conserved region of FXN |
| LGG | | | protein; |
| | | | Common to both TAT-GG-FXN and |
| | | | cynomolgus monkey FXN; |
| | | | Can be used to quantify the endogenous |
| | | | cynomolgus monkey FXN |

In some aspects, the methods of the present invention comprise detecting and/or quantifying at least one of the peptides as listed in Table 2. In some aspects, the methods of the present invention comprise tandem mass spectrometry analysis (MS/MS). For example, in some embodiments, the linker peptide (peptide GGM) may be detected and/or quantified using MS/MS by monitoring the transition 439.2→632.3 and/or the transition 439.2→446.3. In some aspects, the peptide SGT may be detected and/or quantified using MS/MS by monitoring the transition 607.3→669.3. In some aspects, the peptide LGG may be detected and/or quantified using MS/MS by monitoring the transition 625.3→794.4.

In some aspects, the methods of the present invention may involve carrying out liquid chromatography (LC) prior to mass spectrometry. For example, the LC may be a reversed-phase LC.

In some embodiments, the methods of the present invention may involve purifying by immunocapture the unprocessed FXN fusion protein from said sample prior to MS. For example, a method of the present disclosure may comprise the following steps:
a) purifying by immunocapture the unprocessed FXN fusion protein from the sample, thereby obtaining immunocaptured complexes comprising the unprocessed FXN fusion protein;
b) subjecting said immunocaptured complexes comprising the unprocessed FXN fusion protein to digestion by a protease, e.g., trypsin, thereby producing, e.g., the peptide comprising SEQ ID NO: 12; and/or the peptide comprising SEQ ID NO: 13; and/or the peptide comprising SEQ ID NO: 14; and
c) determining the amount of the peptide comprising SEQ ID NO: 12; and/or the peptide comprising SEQ ID NO: 13; and/or the peptide comprising SEQ ID NO: 14 by liquid chromatography and tandem mass spectrometry (LC/MS-MS).

In some embodiments, the peptides produced by said methods include the peptide consisting of SEQ ID NO: 12; and/or the peptide consisting of SEQ ID NO: 13; and/or the peptide consisting of SEQ ID NO: 14. In some embodiments, the amount of peptide determined by liquid chromatography and tandem mass spectrometry (LC/MS-MS) in said method include the peptide consisting of SEQ ID NO: 12; and/or the peptide consisting of SEQ ID NO: 13; and/or the peptide consisting of SEQ ID NO: 14.

In the context of the present invention, the immunocapture may be carried out using a binding protein, e.g., an antibody, comprising an antigen binding domain that specifically binds FXN or FXN fusion protein. In some embodiments, the binding protein, e.g., an antibody, comprises an antigen binding domain that specifically binds to FXN or the FXN portion of the FXN fusion protein.

In some embodiments, the binding protein, e.g., an antibody, comprises an antigen binding domain that specifically binds to the FXN fusion protein but does not bind to FXN, such as endogenous FXN present in a sample. For example, in some embodiments the binding protein, e.g., an antibody, comprises an antigen binding domain that specifically binds to a CPP present in the FXN fusion protein, such as HIV-TAT.

In some aspects, the present disclosure provides a method for determining the amount of unprocessed frataxin (FXN) fusion protein in a sample, the method comprising determining the amount of a peptide in said sample by using mass spectrometry (MS), wherein said peptide comprises the amino acid sequence GGMWTLGR (SEQ ID NO: 12); and wherein the unprocessed FXN fusion protein comprises, from N-terminus to C-terminus, a cell penetrating peptide (CPP); a mitochondrial targeting sequence (MTS) comprising SEQ ID NO: 3; and FXN comprising SEQ ID NO: 2.

In some embodiments, the CPP comprises HIV-TAT (SEQ ID NO: 4) or HIV-TAT+M (SEQ ID NO: 15). In some embodiments, the unprocessed FXN fusion protein comprises the amino acid sequence of SEQ ID NO: 11. In some embodiments, the unprocessed FXN fusion protein consists of the amino acid sequence of TAT-GG-FXN (SEQ ID NO: 11).

In some embodiments, the present disclosure provides a method for determining the amount of unprocessed TAT-GG-FXN (SEQ ID NO: 11) in a sample, the method comprising determining the amount of at least one peptide in said sample by using MS, wherein the at least one peptide is selected from the group consisting of: a) a peptide comprising or consisting of the amino acid sequence SGTLGHPGSLDETTYER (SEQ ID NO: 13); and b) a peptide comprising or consisting of the amino acid sequence LGGDLGTYVINK (SEQ ID NO: 14); wherein the method comprises purifying by immunocapture the unprocessed TAT-GG-FXN from said sample prior to MS; wherein the immunocapture is carried out using a binding protein, e.g., an antibody, comprising an antigen binding domain, wherein said antigen binding domain is capable of binding a TAT protein transduction domain. In some embodiments, the binding protein, e.g., antibody, comprises an antigen binding domain that specifically binds to TAT protein transduction domain present in TAT-GG-FXN.

In some aspects, the present disclosure provides a method for determining the amount of FXN in a tissue sample, the method comprising determining the amount of at least one peptide derived from said FXN in said sample by using MS, wherein said at least one peptide is selected from the group consisting of: a) a peptide comprising or consisting of the amino acid sequence SGTLGHPGSLDETTYER (SEQ ID NO: 13); and b) a peptide comprising or consisting of the amino acid sequence LGGDLGTYVINK (SEQ ID NO: 14); wherein the method is characterized by at least one of the following: the method has a precision as measured by % coefficient of variation (% CV) of 20% or less; and the method has an accuracy of about 80% to about 120%.

### Samples for Analysis Using Methods of the Disclosure

Any sample may be used with the methods of the present disclosure to determine the amount of FXN or FXN fusion protein, e.g., TAT-GG-FXN (SEQ ID NO: 11). In some embodiments, the methods of the present disclosure are useful for analyzing the amount of FXN or FXN fusion protein in a solid sample, e.g., tissue sample, such as a buccal swab or a skin biopsy. In some embodiments, the methods of the present disclosure are useful for analyzing the amount of FXN or FXN fusion protein in a sample derived from blood and may comprise, e.g., platelets. In some embodiments, the sample may be a plasma sample.

Buccal swabs may be obtained from a subject, e.g., a mammal, e.g., a monkey or a human, by collecting buccal cells using Isohelix Swabs. The buccal swabs can be stored in frozen tubes until further use. The buccal swabs may further be processed by addition of a buffer, such as a RIPA buffer. In some embodiments, the RIPA buffer may also contain a protease inhibitor, such as EDTA free Halt™ protease inhibitor cocktail. The buccal swabs may further be vortexed and sonicated prior to immunocapture.

Skin biopsies may be obtained from a subject, e.g., a mammal, e.g., a monkey or a human, by collecting biopsy punches. The biopsy punches may be snap frozen in liquid nitrogen until further use. The skin biopsies may further be processed by mincing the skin punches into pieces, e.g., by using a surgical blade, and transferring the minced tissue to Lysing Matrix D tubes (MP Biomedicals). The skin biopsies may further be processed by addition of a buffer, such as a RIPA buffer. In some examples, the RIPA buffer may also contain a protease inhibitor, such as EDTA free Halt™ protease inhibitor cocktail. The skin biopsies may further be sonicated and homogenized, e.g., by using the FastPrep-96™ grinder. The sonication and homogenization steps may be repeated as necessary prior to immunocapture.

In some embodiments, methods of the present disclosure are particularly suitable for analyzing solid samples, e.g., tissue samples, such as buccal swabs or skin biopsies, and are characterized by higher accuracy and precision as compared to the previously described methods for determining the amount of FXN. For example, the previously described methods of determining the amount of FXN in samples, e.g., solid tissues, such as buccal swabs, were characterized by high variability.

Further, skin samples were not previously used for measuring FXN levels in a subject. The present inventors unexpectedly and surprisingly discovered that FXN may be reliably measured in skin samples. The present inventors have also unexpectedly and surprisingly discovered that levels of FXN or FXN fusion protein, such as TAT-GG-FXN (SEQ ID NO: 11), in the skin, detectably increase as a result of administration of FXN protein replacement therapy, e.g., therapy comprising administration of TAT-GG-FXN. Thus, the present inventors have discovered that skin samples, such as skin biopsies, may be used to monitor levels of FXN in a subject, e.g., following administration of FXN or FXN fusion protein, such as TAT-GG-FXN.

Accordingly, in some aspects, the present disclosure provides a method for determining the amount of FXN in a skin sample, the method comprising determining the amount of at least one peptide derived from said FXN in the skin sample by using MS, wherein the at least one peptide is selected from the group consisting of: a) a peptide comprising the amino acid sequence GGMWTLGR (SEQ ID NO: 12); b) a peptide comprising the amino acid sequence SGTLGHPGSLDETTYER (SEQ ID NO: 13); and c) a peptide comprising the amino acid sequence LGGDLGTYVINK (SEQ ID NO: 14). In some embodiments, the present disclosure provides a method for determining the amount of FXN in a skin sample, the method comprising determining the amount of at least one peptide derived from said FXN in the skin sample by using MS, wherein the at least one peptide is selected from the group consisting of: a) a peptide consisting of the amino acid sequence GGMWTLGR (SEQ ID NO: 12); b) a peptide consisting of the amino acid sequence SGTLGHPGSLDETTYER (SEQ ID NO: 13); and c) a peptide consisting of the amino acid sequence LGGDLGTYVINK (SEQ ID NO: 14).

### EXAMPLES

### Example 1

The goal of this experiment was to develop an LC/MS/MS assay to determine the amount of TAT-GG-FXN (SEQ ID NO: 11) in buccal cells, skin biopsies and platelets derived from cynomolgus monkeys, following repeated administration of TAT-GG-FXN for 14 days.

### Materials and Methods

### Biological Samples

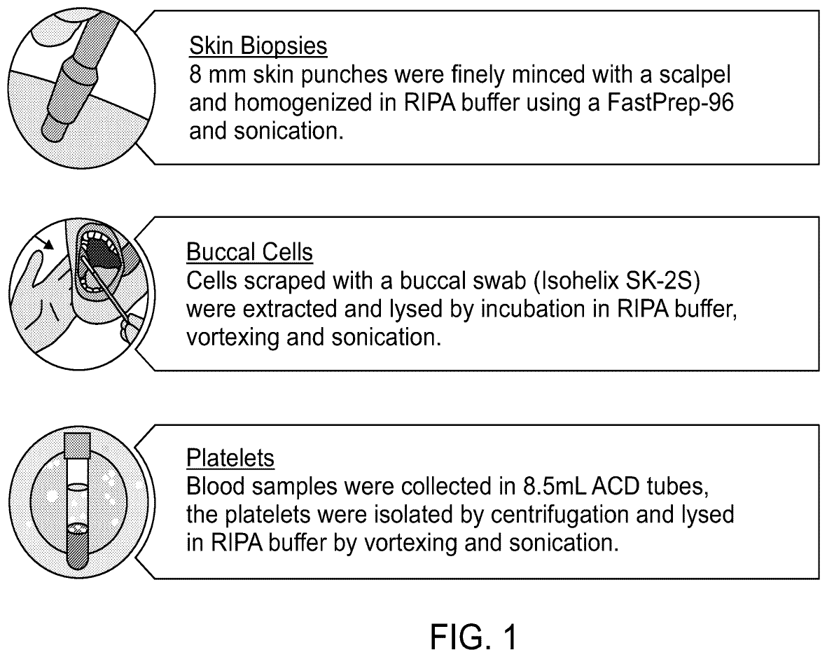

Preparation of biological samples used in the experiment is illustrated in FIG. 1.

Skin biopsy samples were prepared by mincing 8 mm skin punches with a scalpel and homogenizing them in RIPA buffer using a FastPrep-96 and sonication. Buccal cell samples were prepared by extracting cells scraped with a buccal swab (Isohelix SK-2S), lysing by incubation in RIPA buffer, vortexing and sonication. Platelet samples were prepared by collecting blood samples in 8.5 mL ACD tubes, isolating the platelets by centrifugation and lysing in RIPA buffer by vortexing and sonication.

Calibration standards and QCs were prepared in proxy matrix fortified with TAT-GG-FXN from 0.250 to 25.000 ng/mL. Matrix QCs were prepared in pooled tissue homogenates at low and high QC concentrations.

### Total Protein Determination

Total protein concentration of each sample was determined using the Pierce™ BCA Protein Assay Kit and used for data normalization.

### Sample Processing

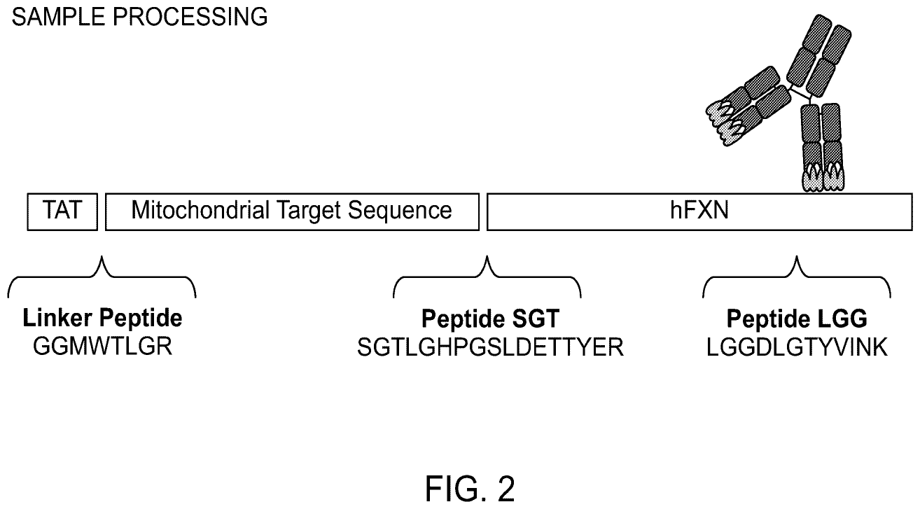

Sample processing for hybrid LC-MS/MS analysis is illustrated in FIG. 2. TAT-GG-FXN was immunopurified using a biotinylated anti-FXN antibody. A 15N-labeled SILAC TAT-GG-FXN was used as an internal standard. Following trypsin digestion, 3 peptides as shown in the Table 4 below and corresponding 15N-labeled peptides were monitored by LC-MS/MS on a SCIEX 6500+.

TABLE 4
Peptides monitored for TAT-GG-FXN quantitation

| Peptide | | SEQ ID | |
|---|---|---|---|
| Name | Amino Acid Sequence | NO. | Characteristics |
| Linker | GGMWTLGR | 12 | Unique to TAT-GG-FXN |
| Peptide | | | Linker between TAT and MTS |
| (Peptide | | | sequences |
| GGM) | | | Not present in TAT-GG-FXN |
| | | | mature form |
| | | | Monitored for informative purpose |
| Peptide | SGTLGHPGSLDETTYER | 13 | N-terminal peptide of the mature |
| SGT | | | TAT-GG-FXN |
| | | | Not found in the monkey proteome |
| | | | Surrogate peptide for TAT-GG-FXN |
| | | | quantitation |
| Peptide | LGGDLGTYVINK | 14 | Peptide from conserved region of |
| LGG | | | FXN protein |
| | | | Common to both TAT-GG-FXN and |
| | | | cynomolgus monkey FXN |
| | | | Can be used to quantify the |
| | | | endogenous cynomolgus monkey |
| | | | FXN |
| | | | Monitored for informative purpose |

### Processing of Results

Peak area ratios of TAT-GG-FXN derived tryptic peptides and their corresponding ¹⁵N-labeled peptides were used to construct calibration curves (weighted 1/x² linear regression).

The concentration of TAT-GG-FXN in samples was determined (in ng/ml) using the calibration curve equation. After normalization using total protein concentration (μg protein per mL of homogenate), the TAT-GG-FXN concentrations are reported as pg of TAT-GG-FXN per μg of total protein.

### Results

Shown in Table 5 below are the results of TAT-GG-FXN measurements in buccal cells, skin biopsies and platelets. Also shown in Table 5 are the precision and accuracy (multi-peptide) of the assay.

TABLE 5
TAT-GG-FXN precision and accuracy (multi-peptide) in
buccal cells, skin biopsies and platelets.

| | | | Nominal | | | |
|---|---|---|---|---|---|---|
| | | | Concentration | | % CV | |
| Peptide | Sample Type | | (ng/mL) | Accuracy | (N = 3) | Code |
| Linker Peptide | Buccal | Low QC | 0.750 | 97.4 | 2.3 | |
| (Peptide GGM) | Cells | High QC | 18.750 | 101.9 | 1.3 | |
| | Skin | Low QC | 0.750 | 99.7 | 8.0 | |
| | Biopsies | High QC | 18.750 | 104.5 | 4.6 | |
| | Platelets | Low QC | 0.750 | 97.5 | 11.1 | |
| | | High QC | 18.750 | 106.4 | 4.4 | |
| Peptide SGT | Buccal | Low QC | 0.750 | 84.4 | 4.3 | |
| | Cells | High QC | 18.750 | 102.6 | 1.2 | |
| | Skin | Low QC | 0.750 | 87.8 | 9.0 | |
| | Biopsies | High QC | 18.750 | 107.1 | 3.1 | |
| | Platelets | Low QC | 0.750 | 92.7 | 7.1 | |
| | | High QC | 18.750 | 107.3 | 2.5 | |
| Peptide LGG | Buccal | Endogenous | 4.869* | 100.0 | 3.2 | |
| | Cells | Low QC | 5.619** | 95.6 | 3.7 | |
| | | High QC | 23.619** | 102.0 | 2.2 | |
| | Skin | Endogenous | 10.153* | 100.0 | 5.0 | |
| | Biopsies | Low QC | 10.903** | 99.4 | 2.8 | |
| | | High QC | 28.903** | 104.4 | 2.3 | >ULOQ |
| | Platelets | Endogenous | 19.867* | 100.0 | 2.2 | |
| | | Low QC | 20.617** | 94.5 | 3.8 | |
| | | High QC | 38.617** | 104.1 | 1.1 | >ULOQ |

>ULOQ: Above upper limit of quantitation. Extrapolated values are presented.
*Back-calculated endogenous level. Average of 3 replicates.
**Endogenous level + spiked-in TAT-GG-FXN at low and high QC levels.

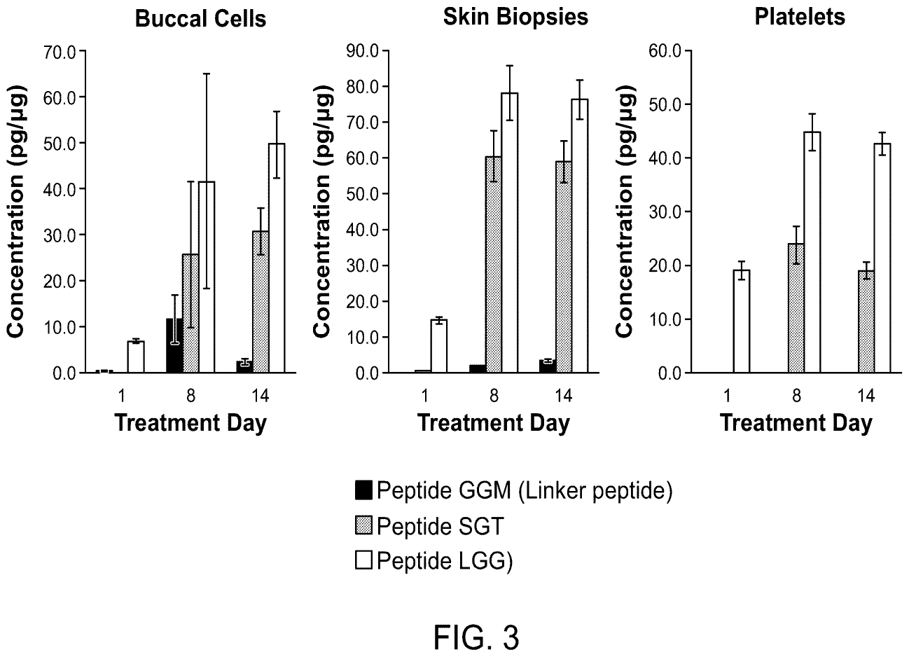

FIG. 3 is a series of graphs showing the concentration of TAT-GG-FXN levels in monkey tissues (N=6) following repeated subcutaneous (SC) administration at 15 mg/kg BID for 14 days. Data is expressed at pg of TAT-GG-FXN per μg of total protein.

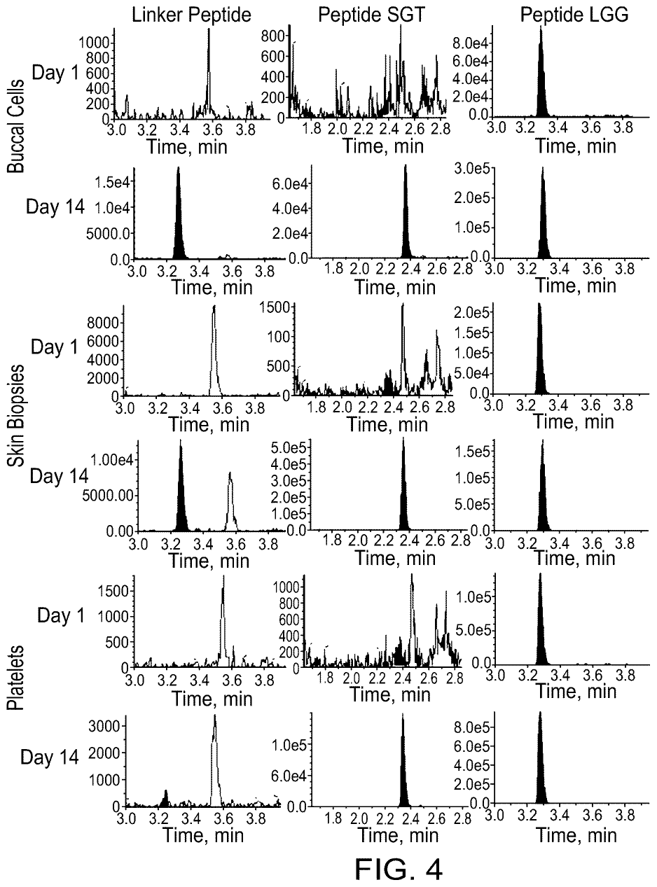

FIG. 4 is a series of representative chromatograms of TAT-GG-FXN tryptic peptides in monkey tissues prior to dosing (Day 1) and following repeated SC administration at 15 mg/kg BID for 14 days.

### CONCLUSIONS

The results from the studies provided in this Example indicate that, following repeated administration of TAT-GG-FXN (SEQ ID NO: 11) for 14 days, TAT-GG-FXN not only accumulates in the tissues outside of the systemic circulation but also is predominantly present as the mature FXN protein. The results from this study illustrate how hybrid LC-MS/MS assays can be used to simultaneously gain insight into the concentration, disposition and processing of biotherapeutics, and in particular, of TAT-GG-FXN.

1. A method for determining the amount of unprocessed frataxin (FXN) fusion protein in a sample, said method comprising determining the amount of a peptide in said sample by using mass spectrometry (MS),
wherein said peptide comprises the amino acid sequence GGMWTLGR (SEQ ID NO: 12); and
wherein said unprocessed FXN fusion protein comprises, from N-terminus to C-terminus, a cell penetrating peptide (CPP); a mitochondrial targeting sequence (MTS) comprising SEQ ID NO: 3; and FXN comprising SEQ ID NO: 2.

2. The method of claim 1, wherein said sample is obtained from a subject being administered the unprocessed FXN fusion protein.

3. The method of claim 1, wherein said CPP comprises HIV-TAT (SEQ ID NO: 4) or HIV-TAT+M (SEQ ID NO: 15).

4. The method of claim 2, wherein said unprocessed FXN fusion protein is TAT-GG-FXN (SEQ ID NO: 11).

5. The method of any one of claims 1-4, wherein said method comprises purifying by immunocapture the unprocessed FXN fusion protein from said sample prior to MS.

6. The method of any one of claims 1-5, wherein said method comprises the following steps:
a) purifying by immunocapture the unprocessed FXN fusion protein from said sample, thereby obtaining immunocaptured complexes comprising the unprocessed FXN fusion protein;
b) subjecting said immunocaptured complexes comprising the unprocessed FXN fusion protein to digestion by trypsin, thereby producing the peptide comprising SEQ ID NO: 12; and
c) determining the amount of the peptide comprising SEQ ID NO: 12 by liquid chromatography and tandem mass spectrometry (LC/MS-MS).

7. The method of claim 6, wherein in part c) the liquid chromatography (LC) comprises reversed-phase chromatography.

8. The method of claim 6 or 7, wherein tandem mass spectrometry (MS-MS) comprises monitoring the transition 439.2→632.3.

9. The method of claim 6 or 7, wherein tandem mass spectrometry (MS-MS) comprises monitoring the transition 439.2→446.3.

10. The method of any one of claims 5-9, wherein the immunocapture is carried out using a binding protein comprising an antigen binding domain that specifically binds to the FXN fusion protein.

11. The method of claim 10, wherein said binding protein specifically binds to the FXN present in the FXN fusion protein.

12. The method of claim 10, wherein said binding protein specifically binds to the CPP present in the FXN fusion protein.

13. The method of claim 12, wherein the FXN fusion protein comprises HIV-TAT (SEQ ID NO: 4) or HIV-TAT+M (SEQ ID NO: 15) and wherein said binding protein specifically binds to the HIV-TAT.

14. The method of any one of claims 1-13, wherein the sample is a tissue sample.

15. The method of claim 14, wherein the tissue sample comprises a buccal swab.

16. The method of claim 14, wherein the tissue sample comprises a skin biopsy.

17. The method of any one of claims 1-12, wherein the sample is derived from the blood of the subject.

18. The method of claim 16, wherein the sample comprises platelets.

19. The method of claim 16, wherein the sample is a plasma sample.

20. The method of any one of claims 1-17, wherein the method has a precision as measured by % coefficient of variation (% CV) of about 20% or less.

21. The method of claim 20, wherein the method has a precision as measured by % CV of about 15% or less, about 14% or less, about 13% or less, about 12% or less, about 11% or less, about 10% or less, about 9% or less, about 8% or less, about 7% or less, about 6% or less, about 5% or less, about 4% or less, about 3% or less, about 2% or less or about 1% or less.

22. The method of any one of claims 1-21, wherein the method has an accuracy of about 80% to about 120%.

23. The method of any one of claims 1-22, wherein the method is linear for concentration of unprocessed FXN fusion protein ranging from about 0.250 ng/ml to about 25.000 ng/mL.

24. A method of monitoring processing of unprocessed FXN fusion protein in a sample, said method comprising:
obtaining at least two temporally separated samples from said subject; and
determining the amount of unprocessed FXN fusion protein in said temporally separated samples using the method of any one of claims 1-23.

25. The method of claim 24, wherein the sample is derived from a subject being administered the unprocessed FXN fusion protein.

26. A method of adjusting a dosing schedule for administering unprocessed FXN fusion protein, said method comprising:
obtaining at least two temporally separated samples from the subject being administered unprocessed FXN fusion protein;
determining the amount of unprocessed FXN fusion protein in said temporally separated samples using the method of any one of claims 1-23; and
adjusting the dose of unprocessed FXN fusion protein being administered to the subject and/or dosing frequency of the unprocessed FXN fusion protein being administered to the subject based on the temporal changes in the amount of unprocessed FXN fusion protein observed in the subject.

27. A method for determining the amount of unprocessed TAT-GG-FXN (SEQ ID NO: 11) in a sample, said method comprising determining the amount of at least one peptide in said sample by using MS,
wherein said at least one peptide is selected from the group consisting of:
a) a peptide comprising the amino acid sequence SGTLGHPGSLDETTYER (SEQ ID NO: 13); and
b) a peptide comprising the amino acid sequence LGGDLGTYVINK (SEQ ID NO: 14);
wherein said method comprises purifying by immunocapture the unprocessed TAT-GG-FXN from said sample prior to MS;
wherein said immunocapture is carried out using a binding protein comprising an antigen binding domain, wherein said binding protein is capable of binding a TAT protein transduction domain.

28. The method of claim 27, wherein said sample is obtained from a subject being administered the unprocessed TAT-GG-FXN.

29. The method of claim 27 or 28, wherein said method comprises the following steps:
a) purifying by immunocapture the unprocessed TAT-GG-FXN from said sample, thereby obtaining immunocaptured complexes comprising the unprocessed TAT-GG-FXN;
b) subjecting said immunocaptured complexes comprising the unprocessed TAT-GG-FXN to digestion by trypsin, thereby producing the peptide comprising SEQ ID NO: 13 and/or the peptide comprising SEQ ID NO: 14; and
c) determining the amount of the peptide comprising SEQ ID NO: 13 and/or the peptide comprising SEQ ID NO: 14 by LC/MS-MS.

30. The method of claim 29, wherein in part c) the LC comprises reversed-phase chromatography.

31. The method of claim 29 or 30, wherein the MS-MS comprises monitoring the transition 607.3→669.3 for the peptide comprising SEQ ID NO: 13 and the transition 625.3→794.4 for the peptide comprising SEQ ID NO: 14.

32. The method of any one of claims 27-31, wherein the sample is a tissue sample.

33. The method of claim 32, wherein the tissue sample comprises a buccal swab.

34. The method of claim 32, wherein the tissue sample comprises a skin biopsy.

35. The method of any one of claims 27-31, wherein the sample is derived from the blood of the subject.

36. The method of claim 35, wherein the sample comprises platelets.

37. The method of claim 35, wherein the sample is a plasma sample.

38. The method of any one of claims 27-37, wherein the method has a precision as measured by % coefficient of variation (% CV) of 20% or less;

39. The method of any one of claims 27-38, wherein the method has an accuracy of about 80% to about 120%.

40. The method of any one of claims 27-39, wherein the method is linear for concentrations of unprocessed TAT-GG-FXN ranging from about 0.250 ng/ml to about 25.000 ng/mL.

41. A method for determining the amount of FXN in a tissue sample, said method comprising determining the amount of at least one peptide derived from said FXN in said sample by using MS,
wherein said at least one peptide is selected from the group consisting of:
a) a peptide comprising the amino acid sequence SGTLGHPGSLDETTYER (SEQ ID NO: 13); and
b) a peptide comprising the amino acid sequence LGGDLGTYVINK (SEQ ID NO: 14);
wherein the method is characterized by at least one of the following:
the method has a precision as measured by % coefficient of variation (% CV) of 20% or less; and
the method has an accuracy of about 80% to about 120%.

42. The method of claim 41, wherein said sample is obtained from a subject.

43. The method of claim 42, wherein said subject is being administered unprocessed FXN fusion protein.

44. The method of claim 43, wherein said unprocessed FXN fusion protein comprises, from N-terminus to C-terminus, a cell penetrating peptide (CPP), a mitochondrial targeting sequence (MTS) of SEQ ID NO: 3 and FXN.

45. The method of claim 44, wherein said CPP comprises HIV-TAT (SEQ ID NO: 4) or HIV-TAT+M (SEQ ID NO: 15).

46. The method of claim 45, wherein said unprocessed FXN fusion protein is TAT-GG-FXN (SEQ ID NO: 11).

47. The method of any one of claims 41-46, wherein said method comprises purifying by immunocapture the FXN from said sample prior to MS.

48. The method of any one of claims 41-47, wherein said method comprises the following steps:
a) purifying by immunocapture the FXN from said sample, thereby obtaining immunocaptured complexes comprising FXN;
b) subjecting said immunocaptured complexes comprising FXN to digestion by trypsin, thereby producing the peptide comprising SEQ ID NO: 13 and/or the peptide comprising SEQ ID NO: 14; and
c) determining the amount of at least one peptide selected from the group consisting of the peptide comprising SEQ ID NO: 13 and/or the peptide comprising SEQ ID NO: 14 by LC/MS-MS.

49. The method of claim 48, wherein in part c) the LC comprises reversed-phase chromatography.

50. The method of claim 48 or 49, wherein the tandem mass spectrometry (MS-MS) comprises monitoring the transition 607.3→669.3 for the peptide comprising SEQ ID NO: 13 and the transition 625.3→794.4 for the peptide comprising SEQ ID NO: 14.

51. The method of any one of claims 47-50, wherein the immunocapture is carried out using a binding protein comprising an antigen binding domain that specifically binds FXN.

52. The method of any one of claims 47-50, wherein said FXN is a part of an FXN fusion protein also comprising HIV-TAT (SEQ ID NO: 4) or HIV-TAT+M (SEQ ID NO: 15) and wherein said binding protein specifically binds the HIV-TAT.

53. The method of any one of claims 41-52, wherein the tissue sample comprises a buccal swab.

54. The method of any one of claims 41-52, wherein the tissue sample comprises a skin biopsy.

55. A method for determining the amount of FXN in a skin sample, said method comprising determining the amount of at least one peptide derived from said FXN in said skin sample by using MS,
wherein said at least one peptide is selected from the group consisting of:
a) a peptide comprising the amino acid sequence GGMWTLGR (SEQ ID NO: 12);
b) a peptide comprising the amino acid sequence SGTLGHPGSLDETTYER (SEQ ID NO: 13); and
c) a peptide comprising the amino acid sequence LGGDLGTYVINK (SEQ ID NO: 14).

56. The method of claim 55, wherein said skin sample is obtained from a subject.

57. The method of claim 56, wherein said subject is being administered unprocessed FXN fusion protein.

58. The method of claim 57, wherein said unprocessed FXN fusion protein comprises, from N-terminus to C-terminus, a cell penetrating peptide (CPP), a mitochondrial targeting sequence (MTS) of SEQ ID NO: 3 and FXN.

59. The method of claim 58, wherein said CPP comprises HIV-TAT (SEQ ID NO: 4) or HIV-TAT+M (SEQ ID NO: 15).

60. The method of claim 59, wherein said unprocessed FXN fusion protein is TAT-GG-FXN (SEQ ID NO: 11).

61. The method of any one of claims 55-60, wherein said method comprises purifying by immunocapture the FXN from said skin sample prior to MS.

62. The method of any one of claims 55-61, wherein said method comprises the following steps:
a) purifying by immunocapture FXN from said skin sample, thereby obtaining immunocaptured complexes comprising FXN;
b) subjecting said immunocaptured complexes comprising FXN to digestion by trypsin, thereby producing the peptide comprising SEQ ID NO: 12 and/or the peptide comprising SEQ ID NO: 13 and/or the peptide comprising SEQ ID NO: 14; and
c) determining the amount of at least one peptide selected from the group consisting of the peptide comprising SEQ ID NO: 12 and/or the peptide comprising SEQ ID NO: 13 and/or the peptide comprising SEQ ID NO: 14 by LC/MS-MS.

63. The method of any one of claims 61-62, wherein said immunocapture is carried out using a binding protein comprising an antigen binding domain that specifically binds FXN.

64. The method of any one of claims 61-63, wherein said FXN is a part of an FXN fusion protein also comprising HIV-TAT (SEQ ID NO: 4) or HIV-TAT+M (SEQ ID NO: 15) and wherein said binding protein specifically binds the HIV-TAT.
